(19) United States
(12) Patent Application Publication
(10) Pub. No.: US 2009/0234121 A1
(43) Pub. Date: Sep. 17, 2009

(54) TRIDENTATE (NNC) CATALYSTS FOR THE SELECTIVE OXIDATION OF HYDROCARBONS

(76) Inventors: Roy A. Periana, Jupiter, FL (US); William A. Goddard, III, Pasadena, CA (US); Jonas Oxgaard, Monrovia, CA (US); Kenneth Young, Los Angeles, CA (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 12/354,409

(22) Filed: Jan. 15, 2009

Related U.S. Application Data
(60) Provisional application No. 61/021,605, filed on Jan. 16, 2008.

(51) Int. Cl. C07C 51/255; C07F 17/02; C07C 15/04; C07C 9/04; C07C 37/58; C07C 29/50; C07C 53/00

(52) U.S. Cl. 546/4; 585/400; 585/700; 568/802; 568/910; 562/408; 562/512.2

(57) ABSTRACT

The synthesis of discrete, air, protic, and thermally stable transition metal NNC complexes that catalyze the CH activation and functionalization of alkanes and arenes is disclosed. Methods for the selective conversion of methane to methanol or methyl esters in acidic and neutral media are disclosed.

### CROSS-REFERENCE TO RELATED APPLICATIONS

The present Application claims priority from U.S. Provisional Patent Application No. 61/021,605 filed Jan. 16, 2008, entitled “Iridium NNC Pincer Complexes,” which is incorporated by reference herein in its entirety.

### BACKGROUND OF THE INVENTION

The direct conversion of natural gas or methane to liquid fuels such methanol or methyl esters promises to expand raw feedstock sources for the petroleum and energy industries. However, the current technology capable of converting methane to methyl esters in high conversion (generating>1M concentrations) and activity is based on electrophilic Pt(II) and Hg(II) CH activation catalysts that require strong acid solvents such as sulfuric acid (H₂SO₄) in order to work efficiently. Such electrophilic catalysts are active in concentrated sulfuric acid because the energy barrier for coordination of methane is low enough to allow CH activation to proceed below 250° C. However, as the acidity of the solvent decreases due to water or methanol formation as the reaction proceeds, so does catalyst activity, and CH activation effectively stops below about ˜85% H₂SO₄. In order to develop the next generation of hydrocarbon oxidation catalysts, new thermally, acidic, and oxidant stable catalysts will be required where the CH activation reaction is not inhibited by water or products.

### SUMMARY OF THE INVENTION

The invention relates to catalysts and catalytic methods for converting alkane and arene hydrocarbons into derivatized products. One embodiment of the invention is the synthesis of air, acid, and thermally stable transition metal complexes having a tridentate NNC ligand that binds and activates a transition metal. Another aspect of the invention is the activation of alkane and arene hydrocarbon CH bonds using of air, acid and thermally stable transition metal NNC complexes in acidic and neutral solvents. Yet another aspect of the invention is the selective oxidation of alkane and arene hydrocarbon CH bonds using of air, acid and thermally stable transition metal NNC complexes in acidic and neutral solvents.

### BRIEF DESCRIPTION OF THE DRAWINGS

FIG. 1 shows a general scheme for converting arene and alkane hydrocarbons (represented by RH) to derivatized products via C—H activation and functionalization.

FIG. 2 shows a diagrammatic scheme for a Wacker type air oxidized process for converting hydrocarbons to useful products.

FIG. 3 shows a computionally determined energy diagram for benzene CH activation with the complex 1-Cl.

FIG. 4 shows an Erying plot for the stoichiometric benzene activation with 1-Cl.

### DETAILED DESCRIPTION OF THE INVENTION

In order to provide a clearer and more consistent understanding of the specification and the claims, the following definitions are provided:

The term “activating” refers in general to causing a chemical species to be reactive with other chemical species. In a non-limiting example, a catalyst which may be normally inactive or slow to react may be activated by the addition or via contact with another agent, where the agent can be a solvent or surrounding environment.

The term “activating a CH bond” refers to a process whereby a CH bond and a metal ligand complex react to generate a metal-alkyl complex. The newly formed methyl-alkyl complex comprises a metal-carbon bond. The reaction comprises two steps that contribute to the energy barrier for the overall reaction. The two steps are (1) CH bond coordination to a metal catalyst and (2), subsequent CH bond cleavage to yield a metal alkyl complex. Theoretical studies suggest that a NNC ligand favorably influences the electronic nature of a ligated metal center, activating the metal catalyst, leading to a reduction in the energetic barrier for both steps (1) and (2). CH activation as defined herein proceeds without the involvement of free radicals, carbocations or carbanions to generate metal alkyl intermediates. CH activation which does not include a complete and irreversible conversion to functionalized alkane products may be physically detected by the incorporation of hydrogen isotopes (deuterium or tritium) into alkane reactant. Thus CH activation may also refer to the ability of a catalyst to catalyze H/D exchange between an alkane or arene reactant and a deuterium source such as for example a deuterated solvent. Other trapping reagents may be used to detect or screen for CH activation.

The term “alkane” refers to non-aromatic saturated hydrocarbons with the general formula CₙH(2ₙ+2), where n is 1 or greater. Alkanes may be straight chained or branched. Examples include methane, ethane, propane, butane (branched and linear), cyclohexane, cyclooctane. Alkanes may be in a solid, liquid or gas phase.

The term “arene” refers to an unsaturated hydrocarbon, the molecular structure of which incorporates one or more planar sets of carbon atoms that are connected by delocalized electrons. A prototype aromatic compound is benzene. Other examples of arenes are polycyclic aromatic hydrocarbons comprising more than one aromatic ring.

The term “catalyst” refers to a substance that initiates or accelerates a chemical reaction without itself being affected. According to several embodiments of the invention, catalysts facilitate chemical reactions between hydrocarbons, functionalization reagents, oxidants, solvents and other components of a chemical transformation. Catalysts themselves are not consumed, rather they are regenerated in situ or in a later recovery step. Coordination catalysts are a class of catalysts that facilitate chemical reactions by bringing together or “coordinating” reactants. Coordination catalyst reactions proceed within the first coordination sphere of an atom of the catalyst. This is as opposed to in the second or other coordination sphere.

The term “catalytic composition” refers to a catalyst and supporting agents such reactants, solvent, functionalization agent, and oxidant.

The term “conjugated π-system” refers to a planar organic compound containing two or more conjugated multiple bonds. Arenes as defined herein have conjugated π-systems. Conjugated π-systems may also comprise hetero atoms and metal atoms.

The term “feed comprising hydrocarbons and an functionalization agent” refers to a mixture of hydrocarbon and a functionalization agent entering a reactor. Feed is consumed by a chemical reaction and the result is a desired chemical product. Feed may be processed to extract a desired product or spent functionalization agent, or a functionalization agent may be recycled.

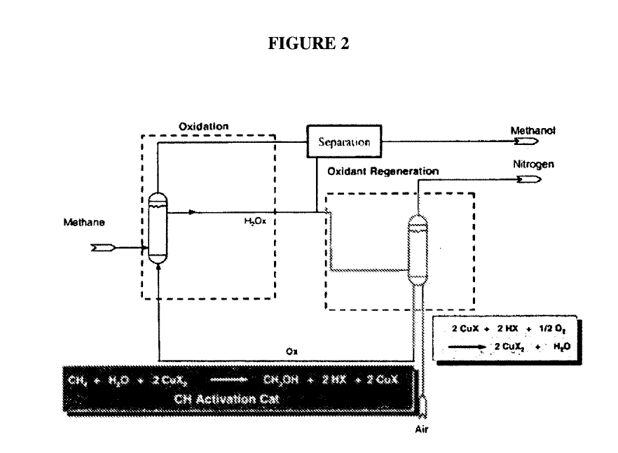

The term “first catalyst zone” refers to a chemical process reactor. Such a first catalyst zone wherein hydrocarbon CH bond activation and functionalization occur is shown schematically in FIG. 2. In FIG. 2, the first catalyst zone is distinct from a regeneration zone where regeneration of the functionalization occurs. FIG. 2 shows a first reactor zone (indication by dashed lines where hydrocarbon oxidation occurs. Here, the term hydrocarbon oxidation refers to hydrocarbon functionalization because oxygen is the atom which replaces the hydrogen in a CH bond. According to FIG. 2, methane feed enters a first catalyst zone comprising an activated metal catalyst of the present invention at functionalization conditions. Also present within the first catalyst zone are solvent or solid support. Also shown in FIG. 2 is a functionalization reagent entering a first catalyst zone. After hydrocarbon functionalization occurs according to the generic reaction equation in FIG. 2, effluent leaving the first catalyst zone comprises functionalized hydrocarbon and depleted functionalization reagent. Functionalized hydrocarbon is separated from depleted functionalization reagent, and depleted functionalization reagent is passed to a regeneration zone to reform the oxidant using air as an oxidant. Reoxidation conditions will vary according to the particular oxidant used in FIG. 2. For CuX/CuX₂ as shown in FIG. 2, the Wacker process is used to reform the oxidant. Other processes which allow the regeneration of functionalization reagent are also considered within the scope of the present invention.

The term “formal anionic ligand” refers to ligands that are anionic, with respect to charge, when formally removed from the metal in their closed shell electronic state. Formal anionic ligands include hydride, halide, C₁-C₆ alkyl, substituted alkyl, alkoxy, carboxylate, bisulfate. Formal anionic ligands also include conjugate bases of neutral protic solvents, for example, hydroxyl (OH⁻), which is the conjugate base of neutral water. Other more specific examples of carboxylate ligands include acetate, halogenated acetate, perhalogentated acetate, including mono, di- and trihaloacetates. Very specific examples of acetate ligands include CH₃C(O)O⁻ and CF₃C(O)O⁻, the conjugate acids of acetic acid and trifluoroacetic acids respectively.

The term “formal neutral ligand” refers to ligands that are formally neutral with respect to charge, when formally removed from the metal in their closed shell electron state. Formal neutral ligands include linear alkenes: ethylene, propylene, 1-butene, 2-butene, 1-pentene, 2-pentene, 1-hexene, 2-hexene, 3-hexene, 1-heptene, 2-heptene, 3-heptene, 1-hexene, 2-hexene, 3-hexene, 4-hexene, cyclic alkenes including cyclohexene, cycloheptene, cyclooctene; branched alkenes including, 2-methyl-1-butene, 2-methyl-2-butene, 3-methyl-1-butene; 2,3-dimethyl-1-butene, 2,3-dimethyl-2-butene, 3,3-dimethyl-1-butene. Other formal neutral ligands L include oxygenated hydrocarbons including without limitation: tetrahydrofuran, 1,4-dioxane, ethylacetate, methylacetate, water (aquo), methyl trifluoroacetate, methanol. Other formal neutral ligands L include nitrogen-containing small molecules including without limitation nitriles, acetonitrile, benzonitrile, tetrafluorobenzonitrile, pentafluorobenzonitrile, pyridine, 2,6-dimethylpyridine.

The term “functionalized hydrocarbon” refers to a hydrocarbon wherein at least one CH bond has been transformed into a carbon functional group bond, a carbon heteroatom bond, where the heteroatom is anything other than H. By way of example only, functionalized methane is methanol. Functionalized benzene is phenol.

The term “functionalization conditions” refers to conditions and components required within a first reactor zone to transform a hydrocarbon into a functionalized hydrocarbon. Functionalization conditions include the type of metal ligand complex, solvent, temperature, and functionalization reagent. In one embodiment the metal is selected from the group consisting of Re, Ru, Os, Rh, and Ir. The oxidation state of suitable metals is intermediary, neither the highest oxidation state, nor metallic. More specifically, oxidation states of the metal are Re(I), Re(II), Re(III), Ru(II), Ru(III), Os(II), Rh(I), Rh(III) and Ir(III).

The term “functionalization” refers to a thermodynamically favorable reaction (ΔG<0) that replaces an H atom of a CH moiety with another atom or moiety, to produce for example, R—OH, R—NH₂, R—Se(O)OH, R—SO₂H, etc.

The term “Group 8 of the periodic table” refers to the elements iron, ruthenium, and osmium.

The term “Group 9 of the periodic table” refers to the elements cobalt, rhodium, and iridium.

The term “hydrocarbon CH bond” refers to a covalent bond between hydrogen and carbon atoms localized within a hydrocarbon molecule. A CH bond may be described in terms of frontier molecular orbital theory as having a highest occupied molecular orbital (HOMO) and a lowest unoccupied molecular orbital (LUMO).

The phrase “hydrocarbon activation is accelerated by solvent” refers to a rate increase due to solvent which is predicted or observed for a CH bond activation event.

The term “ligand” refers to the set of atoms, ions, or molecules in contact with a metal ion. A ligand comprises the set of atoms, ions, or molecules surrounding a metal ion in the first coordination sphere of the metal. Free ligands may be indistinguishable from solvent molecules.

The term “ligating atom” refers to atom or atoms comprised by a ligand which bind to a metal. The term “ligating atom” is equivalent to “donor atom” in certain embodiments.

The term “linked nitrogen heterocycle” refers to bipyridine, bipyrazine, bipyrimidine and the like.

The term “metal-alkyl covalent bond” refers to an alkyl group bonded to a transition metal or metal complex.

The term “metal alkyl complex” refers to an alkyl group bonded to a metal complex.

The term “N-donor atom” refers to ligand or solvent molecules which bind directly to a metal according to certain embodiments of the invention. N-donor atoms may be part of N-donor ligands. Suitable N-donor ligands include nitrogen heterocycles as defined above.

The term “nitrogen heterocycle” refers to organic compounds that contain a ring structure containing nitrogen atoms as part of the ring. They may be either simple aromatic rings or non-aromatic rings. Some examples are pyridine, pyrimidine, and pyrazine.

The term “non-radical producing” refers to a method or process characterized by the absence of free radical. Such radicals may be oxygen-based, halogen based, carbon based, or metal based, including both transition and main group metals.

The term “O-atom donor” refers to any O-atom donor that has a potential to thermodynamically oxidize methane to methanol at a temperature of 300° C. or lower. Thermodynamic potentials for methane oxidation may be calculated from the equation:

CH₄+YO═CH₃H+Y (1)

The change in Gibbs free energy for this reaction, ΔGᵣₓn, determines whether an O-atom transfer donor has the potential to thermodynamically oxidize methane. Values ΔGᵣₓₙ<0 based on calculated or tabulated data for the equation: CH₄+YO═CH₃OH+Y indicate the conversion of methane is feasible, however such reactions which are slightly unfavorable (ΔGᵣₓₙ>0) can be coupled to other reactions to drive them to completion. Approximate values of ΔGᵣₓₙ may be obtained by considering the bond strengths of the reactants and products. On this basis any oxidant (YO) with Y—O bond strength of less than about 90 kcal/mol is a candidate O-atom donor.

The term “N-donor atom” refers to ligand or solvent molecules which bind directly to metals according to certain embodiments of the invention. N-donor atoms may be part of N-donor ligands. Suitable N-donor ligands include nitrogen heterocycles as defined above.

The term “optionally substituted carboxylate” refers to a carboxylate moiety wherein CH bonds of the alkyl portion are substituted. Examples of optionally substituted carboxylates include halogenated carboxylates derived from halogenated carboxylic acids. Examples of halogens include fluorine, chlorine, bromine.

The term “oxidant” (FR, functionalization reagent) refers to a compound (or mixture) that oxidizes (Functionalizes) (removes electrons from. Of courses, if Q in RQ (the functionalized product) is more electronegative than C, the R groups formally “oxidized”.

In general, the reaction R—H+YO->R—OH+Y is a formal atom insertion reaction that is thermodynamically favorable. In the specific case involving oxygen, the atom inserted is an oxygen atom, and the reaction has been termed an “oxidation.” Other examples of the general equation R—H+YO->R—OH+Y include without limitation:

R—H+½O₂->R—OH (2)

R—H+SO₃->R—SO₃H (3)

R—H+H₂N—NH₂->RNH2+H₃N (4)

The functionalization reactions (2)-(4) are all thermodynamically favorable (in Eq (4) when R═C₆H₅) and the H is substituted for OH, SO₃H or NH₂ respectively. In doing so, the oxidizing agent, sometimes called an oxidizer or oxidant or functionalization reagent, becomes reduced (gains electrons).

In equation (1), the generic R—H+YO->R—OH+Y, Y is a depleted functionalization reagent (or reduced oxidant) and cannot react with R—H to substitute H in a thermodynamically favorable reaction (below 250° C.). In the equation (2), O₂ is consumed and becomes R—OH, in equation (3), SO₃ is consumed and becomes R—SO₃H, and in equation (4) hydrazine (N₂H₆) is consumed and becomes NH₃.

An oxidizing chemical reaction is a broadly defined and may have several meanings. In one definition, an oxidizing agent receives (accepts) electrons from another substance (reductant). In this context, the oxidizing agent is called an electron acceptor. Broadly speaking, such chemical events occur in two distinct ways which can be described as inner sphere or outer sphere. In another meaning, an oxidant transfers O atoms to the reductant. In this context, the oxidizing agent can be called an oxygenation reagent or oxygen-atom transfer agent. Examples include amine-N-oxide, cupric oxide, iron oxide, periodate (IO₄⁻), vanadate (VO₄³⁻), molybdate (MoO₄²⁻), nitrous oxide (N₂O), hydrogen peroxide (H₂O₂), selenate (SeO₄²⁻), tellurate (TeO₄²⁻), hypochlorite (ClO⁻), chlorite (ClO₂⁻), nitrate (NO₃⁻), and sulfoxide. NO₂⁻ (nitrite) can also act as an oxidant; e.g. 2 NO₂⁻+CH₄+H₂O->CH₃OH+2OH⁻+2 NO ΔG_{250c}=3 kcal/mol) In this case, Y is NO and can be regenerated by air: NO+¼O₂+HO⁻->NO₂⁻+½ H₂O DG_{250c}=−30 kcal/mol.

The term “oxidation stable solvent” refers to a solvent that is not itself oxidized during any step of a chemical reaction, method, or process.

The term “oxygen insertion agent” refers to an agent that functions as both an oxidant and as a source for an oxygen atom which inserts into a metal-alkyl covalent bond with favorable thermodynamics. Examples include amine-N-oxide, cupric oxide, iron oxide, periodate (IO₄⁻), vanadate (VO₄³⁻), molybdate (MoO₄²⁻), nitrous oxide (N₂O), hydrogen peroxide (H₂O₂), selenate (SeO₄²⁻), tellurate (TeO₄²⁻), hypochlorite (ClO⁻), chlorite (ClO₂⁻), nitrate (NO₃⁻), and sulfoxide.

The term “oxygenated hydrocarbon” refers to a hydroxylated hydrocarbon. Methanol is an oxygenated hydrocarbon (methane).

The term “oxidation resistant ligands” refers a ligand(s) that is not itself oxidized during any step of a chemical reaction, method, or process.

The term “reduced oxidant” refers to an oxidant which has transferred an O atom during or as a consequence of an alkane functionalization process. By way of example, for the oxidant SeO₄²⁻ the reduced oxidant is SeO₃²⁻.

The term “regenerating the catalyst” refers to a step during a process for the selective oxidation of hydrocarbons. During this step, a reduced oxidant or reduced functionalization reagent is reoxidized into an oxidant or a functionalization reagent respectively. Preferred reoxidizing agents are air or dioxygen (O₂). Suitable oxidants are those that can be reoxidized with air in a thermodynamically favorable reaction: Y+½O₂→YO where ΔGᵣₓₙ<0 kcal/mol at temperatures below 300° C. On the basis of tabulated data, the following specific examples are given by way of example only.
SeO4²⁻+CH₄→SeO₃²−+CH₃OH ΔG=−12 kcal/mol at 250° C., K=10⁵
SeO₃²⁻+½ O₂→SeO₄²⁻ΔG=−14 kcal/mol at 250° C., K=10⁵
NO3−+CH₄→NO₂₋+CH₃OH ΔG=−11 kcal/mol at 250° C., K=10⁴
NO₂⁻+½O₂→NO₃⁻ΔG=−15 kcal/mol at 250° C., K=10⁶
CH₃S(O)CH₃+CH4→CH₃OH+CH₃SCH₃ ΔG=−2 kcal/mol at 250° C., K=6
CH₃SCH₃+½ O₂→CH₃S(O)CH₃ ΔG=−17 kcal/mol at 250° C., K=10⁷
2 NO₂⁻+CH₄+H₂O->CH₃OH⁻+2OH⁻+2 NO ΔG_{250c}=3 kcal/mol
NO+¼O₂+HO⁻->NO₂⁻+½H₂O ΔG_{250c}=−30 kcal/mol
   The term “regeneration zone” refers to a second reaction used to regenerate a depleted functionalization reagent. FIG. 2 depicts a regeneration zone according to one embodiment. In FIG. 2, a regeneration zone receives a depleted functionalization reagent which is regenerated using air to oxidant.

The term “regenerating conditions” refers to conditions and components required within a regeneration zone to transform a depleted functionalization reagent back into a functionalization reagent. Regenerating conditions will vary according to the particular functionalization reagent used. For CuX/CuX₂ as shown in FIG. 2, conditions used in the known Wacker process may be used for example to reform the oxidant.

The term “releasing an oxidized hydrocarbon” refers to a step during a process for selectively oxidizing hydrocarbons as disclosed herein. During this step, an oxidized hydrocarbon is released from a metal.

The term “selectively oxidizing” refers to CH bond selectivity exhibited by a catalyst during CH bond activation and subsequent steps. Selective oxidation occurs for example when a catalyst selects a primary versus a secondary or tertiary CH bond. Selectivity can also occur when a catalyst selects an alkyl CH bond of an unreacted hydrocarbon versus that of an oxidized or functionalized hydrocarbon.

The term “solid support” refers to an insoluble matrix to which a catalyst or catalyst complex is attached. An example is an ion exchange resin. Other examples include but are not limited to metal oxides such as magnesium oxide, calcium oxide, and barium oxide as well as potassium fluoride on alumina and some zeolites.

The term “solvent assisted” refers to the role a solvent molecule plays in reaction energetics of a CH bond activating step. A consequence of solvent assistance is an increased reaction rate for a CH bond activating step and an overall hydrocarbon oxidation process.

The term “tridentate ligand catalyst” refers to a catalyst composition wherein a metal center is bound or ligated by three donor atoms which belong to a single ligand entity. An example of a tridentate ligand is 6-phenyl-2,2′-bipyridine, an NNC ligand.

The term “NNC tridentate ligand” refers to which surrounds a metal center in pincer fashion using two nitrogen and a carbon atoms (hence the name NNC). When a metal-free tridentate NNC ligand binds a metal, the hydrogen atom at the 6 position in the phenyl ring is replaced by a bond to the metal atom.

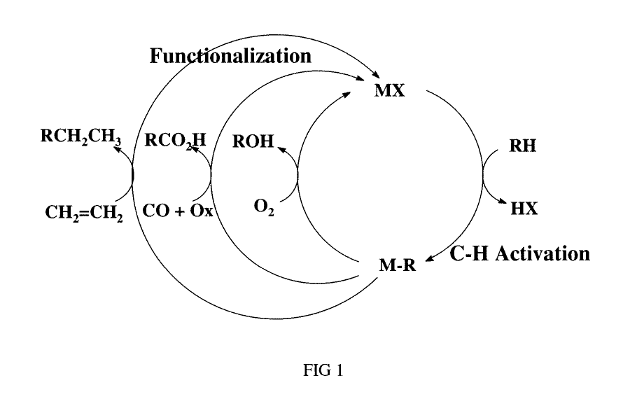

As shown in FIG. 1, CH activation of RH proceeds via the cleavage of the RH bond and generation of a metal alkyl complex, M-R as an intermediate. CH activation may be comprise two discrete steps that contribute to the activation barrier; substrate coordination and CH cleavage. Since breaking the CH bonds of hydrocarbons at lower temperatures leads to catalysts that operate at lower temperatures, minimizing the energy of the two steps involved in breaking the CH bond is important in reducing the activation barrier to the CH activation reaction. Both steps can contribute significantly to the overall barrier and reducing one or both can lead to the generation of efficient catalysts.

The CH activation reaction is useful since this reaction occurs rapidly at lower temperatures, is highly selective and can be coupled with functionalization reactions into catalytic sequences as generally shown in FIG. 1, for the generation of useful products such as alcohols, carboxylic acid, and olefinated products.

Coordination catalysis (including CH activation) is a very efficient form of catalysis because the bond rearrangements of the substrates to products are mediated within the first coordination sphere of another atom or atoms that constitute the catalyst. This is useful because the reactants are “controlled” by the catalyst throughout the transformation since the reactants are bonded to the catalyst. This is in contrast to reactions where the reactants are generated as “free” species with intrinsic reactivity that cannot be controlled, e.g. “free” radical, solvent separated carbocations, carbanions or carbenes. Calculation results carried out on the catalyst of the present invention show that the CH activation steps do not operate by free-radical mechanism. Additionally, since energy is released in making bonds to the catalyst in coordination catalysis, this energy can compensate for the energy required to break strong bonds in the substrate.

Acid and base chemistry are examples of coordination catalysis involving protons and bases, where the chemistry occurs within the coordination sphere of these catalysts. In most acid and base catalyzed reactions, the substrates involved, olefins, carbonyl, arenes, alcohols, etc. are very good coordination species and can readily coordinate to protons or bases. However, a key challenge in hydrocarbon chemistry, especially alkanes, is that these species are among the poorest ligands known. Indeed, while coordination metal complexes of almost all functional classes of molecules are known, stable alkane complexes have not yet been generated. A consequence of this is that efficient coordination catalysis of the alkanes has not yet been developed.

Another challenge to developing coordination catalyst for the conversion of alkanes to alcohols is that alcohols are more coordinating than alkanes. Thus, many coordination catalysts preferentially bind alcohols rather than alkanes. This becomes problematic because the product alcohol can inhibit the alkane conversion catalyst. Alcohols are similar to water in basicity and coordinating capability and alcohols can be readily dehydrated to generate water. Additionally, in many circumstances it is desirable to carry out coordination catalysis in a solvent and in many cases the desirable solvents are protic substances such as water, acids, bases, etc. Consequently, designing catalysts that are not inhibited by water is one of the central challenges to developing catalysts that efficiently oxidize alkanes to alcohols. Catalysts and catalyst compositions of the present invention are water tolerant, and have reduced affinity for water and other nucleophiles, unlike those based on electrophilic metals. Without being bound to theory, the electronic nature of the metal center and NNC ligand both act to reduce attractive interactions between water or product, as well as generating destabilizing interactions (repulsion) between water or product.

One aspect of the present invention are ligands for use with electropositive metals other than Pt(II) and Hg(II). Such electropositive metals include, but are not limited to iridium (Ir), osmium (Os), and rhenium (Re). Such metals are known to exist in various formal oxidation states, whether transiently or as stable isolable complexes. The present invention is not limited to embodiments having any particular or fixed formal oxidation state, insofar as a stable isolable complex may access more than one oxidation state. For example, in one embodiment, an iridium atom of has a formal oxidation state of (III), i.e., Ir(III). Other suitable metals and oxidations states in Os(II) and Re(II). In general, a formal metal oxidation state of (O), that is, zero-valent metals are typically avoided, because such reduced metal atoms tend to aggregate and produce bulk metal.

Metal atoms of the present invention are surrounded by ligands which support and assist the metal atoms and prevent metal-metal aggregation. One generic embodiment are tridentate NNC ligands based on a core structure depicted in structure I.

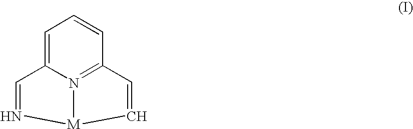

The core elements of structure I include a metal center covalently linked to two nitrogen and a carbon atoms (hence the name NNC) arrayed in a planar or T-shaped geometry. The NNC elements are linked together as shown and feature a delocalized planar π-electron system. Calculational studies show that such an NNC metal complex favorably influences the electronic nature density at a metal center, leading to a reduction in the energetic barrier for both binding (coordination) and cleavage of an alkane CH bond. The later chemical events are important features of a working catalyst and catalytic systems. DFT calculations also predict that an alternative geometry, NCN in which two nitrogen atom donors symmetrically flank a central carbon donor predict higher barriers and thus less active catalysts Tang et al. (PCT publication number WO 2005/120198).

A skilled artisan will appreciate that the basic features of structure I are present in various embodiments of the present invention even though for example, the formamidine moiety (at 9 o'clock in structure I) and the vinylidene moiety (at 3 o'clock in structure I) are subsumed into additional pyridine and phenyl ring moieties respectively. Such an “expansion” of structure I does not represent a departure from the core elements of structure I, but rather represents a working embodiment which may be prepared according to the methods disclosed herein.

A skilled artisan will also recognize that the metal center M in structure I is coordinatively unsaturated, meaning that other ligands (not shown) are typically present to and are closely associated if not chemically bonded to the metal center.

One embodiment of a working catalyst are based on 6-phenyl-2,2′-bipyridine and substituted derivatives thereof shown in structure II.

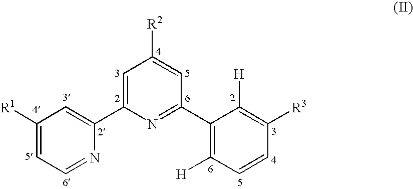

The numbering system used to identifies substituted variants of the parent NNC ligand, where R¹=R²=R³ is H. A ligand is called “NNC” because of the spatial arrangement of 3 atoms (two nitrogens and one carbon) which ligate or covalently a metal atom when a ligand is used to support a metal atom.

One specific embodiment is the NNC ligand, 6-phenyl-4,4′-di-tert-butyl-2,2′-bipyridine, an NNC ligand wherein hydrogen atoms at the 4 and 4′ positions of the two pyridine rings are replaced by electron-donating and more sterically demanding tert-butyl substituents. Suitable substituents include but are not limited to branched alkyl groups. Other suitable substituents include alkoxy substituents for example tert-butoxy isopropoxy, sec-butoxy and the like.

In another embodiment, R¹ and R² are two identical substituents more electron-donating than R³, for example R¹=R²=alkyl and R³═H. In another embodiment, R¹═R²═H, and R³ is a moiety more electron withdrawing than R¹ and R², for example R³ is CF³.

NNC tridentate ligands are used to prepare metal containing tridentate complexes, such for example, a metal complex having structure:

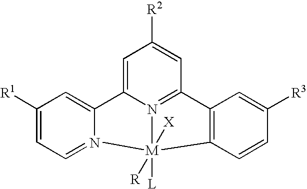

where R¹, R², R³ are each independently H or branched alkyl;
M is a metal more electropositive than platinum, for example iridium, osmium, or rhenium. Example 1 describes the preparation of tridentate NNC compounds for M is iridium.

The tridentate geometry of an NNC ligand surrounds a metal center in pincer fashion and is meridional, i.e., the N,N, and C of the NNC ligand all lie in a single plane. When an NNC ligand binds a metal in a tridentate fashion, the hydrogen atom at the 6 position in the phenyl ring is replaced by a bond to the metal atom.

A transition metal such as iridium in formal oxidation state (III) is usually surrounded by a total of six ligands. Isolated, well characterized embodiments of the invention include NNC complexes comprising Ir(III), and three additional ligands, labeled R, X, and L.

In one embodiment, R is a C₁-C₂ linear alkyl moiety derived from a C₁-C₂ alkane, for example, but not limited to methyl and ethyl, derived from methane, ethane, or ethylene.

In another embodiment, R acts as a base and is a moiety capable of receiving a proton from a hydrocarbon substrate. Other suitable R moieties include R═OH, and NH₂, moieties which impart aqueous solubility to NNC ligands and NNC-metal complexes. Protic groups such as hydroxyl and amine can increase the reactivity of catalysts by undergoing reversible protonation/deprotonation in protic media.

Formal neutral ligands L are defined as ligands that are formally neutral with respect to charge, when formally removed from the metal in their closed shell electron state. Formal neutral ligands include linear alkenes: ethylene, propylene, 1-butene, 2-butene, 1-pentene, 2-pentene, 1-hexene, 2-hexene, 3-hexene, 1-heptene, 2-heptene, 3-heptene, 1-hexene, 2-hexene, 3-hexene, 4-hexene, cyclic alkenes including cyclohexene, cycloheptene, cyclooctene; branched alkenes including, 2-methyl-1-butene, 2-methyl-2-butene, 3-methyl-1-butene; 2,3-dimethyl-1-butene, 2,3-dimethyl-2-butene, 3,3-dimethyl-1-butene.

Other neutral ligands L include oxygenated hydrocarbons including without limitation: tetrahydrofuran, 1,4-dioxane, ethylacetate, methylacetate, water (aquo), methyl trifluoroacetate, methanol;

Other neutral ligands L include nitrogen-containing small molecules including without limitation nitrites: acetonitrile, benzonitrile, tetrafluorobenzonitrile, pentafluorobenzonitrile, pyridine, and 2,6-dimethylpyridine.

Formal anionic ligands X are defined as ligands that are anionic, with respect to charge, when formally removed from the metal in their closed shell electronic state. Formal anionic species include hydride, halide, C₁-C₆ alkyl, substituted alkyl, alkoxy, carboxylate, bisulfate. Formal anionic ligands include conjugate bases of protic solvent, for example, hydroxyl (OH−), the conjugate base of neutral water. Examples of carboxylate ligands include acetate, halogenated acetate, perhalogentated acetate, including mono, di- and trihaloacetates. Specific examples of acetate ligands include CH₃C(O)O⁻ and CF₃C(O)O⁻, the conjugate acids of acetic acid and trifluoroacetic acids respectively.

Formal anionic ligands and formal neutral ligands are interconvertable. For example, a formal neutral aquo ligand (L) can interconvert with a formal anionic ligand X (hydroxy) by loss or gain of a proton. In one embodiment, X is hydroxy (—OH) and L is water (aquo) ligand, under conditions of rapid proton transfer, making X and L indistinguishable. In another example, an anionic carboxylate ligand X can interconvert with a formal neutral carboxylic acid.

In another embodiment, a catalyst comprises an NNC complex having structure:

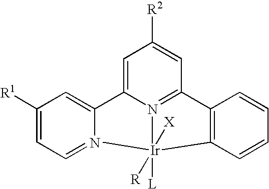

where R¹=R²=tert-butyl;

R is ethyl;

L is alkene or nitrile

X is halide or trifluoroacetate.

In another embodiment, a catalyst comprises an NNC complex having structure:

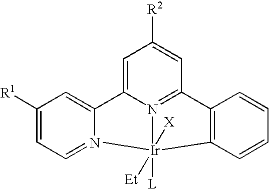

where R¹=R² is tert-butyl;

X═Cl or trifluoroacetate;

L is ethylene or acetonitrile.

Another embodiment are catalyst systems comprising a metal selected from the group consisting of iridium, osmium, and rhenium and an NNC ligand, and certain auxiliary ligands, R, X, and L.

In one embodiment, a tridentate NNC ligand and iridium, Ir(NNC)R(X)(C₂H₄), efficiently activates benzene in acid solvents weaker than sulfuric acid such as acetic acid and trifluoroacetic acid. This catalyst composition is also thermally stable to acidic oxidizing conditions.

Catalysts of the invention are particularly effective in part because systems comprising such catalysts are thermally stable under acidic, oxidizing conditions and also because they operate by a reaction mechanism that does not involve high energy species like free-radicals, carbocations or carbanions, which highly reactive and chemically promiscuous species that compromise selectivity.

The core elements of structure I are embodied in a complex derived from 6-phenyl-2,2′-bipyridine, an NNC ligand lacking alkyl substituents. The complex (NNC)Ir(CF₃CO₂)₂(CF₃CO₂H) is active for CH activation of methane in acetic and CF₃CO₂H. Adding OH groups makes system more soluble in water/base and we have seen activity.

Preparation of an alkyl substituted NNC ligand, 6-phenyl-4,4′-di-tert-butyl, -2,2′-bipyridine and subsequent reaction with [Ir(C₂H₄)₂Cl]₂ led to a tridentate NNC complex trans-Ir(NNC)Cl(C₂H₄)Et complex (1-Cl) as the major product in 66% yield as an air-stable solid according to equation 1. Details of the synthetic procedure are described in Example 1.

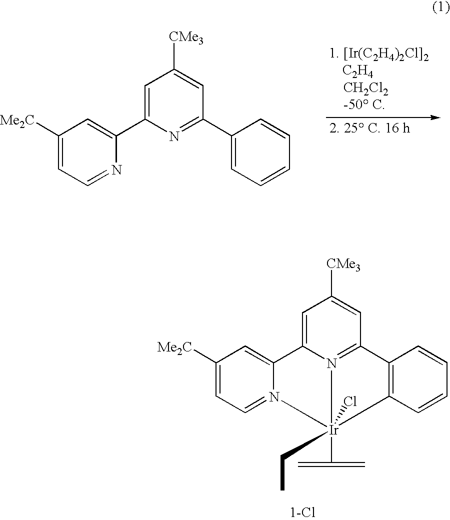

Complex 1-Cl has been fully characterized spectroscopically, including by X-ray crystallography. A second catalyst embodiment, closely related to 1-Cl, is 1-Cl—NCCH₃, wherein an ethylene ligand has been replaced by an acetonitrile moiety. A synthesis of 1-Cl—NCCH₃ from 1-Cl described in Example 1 below.

1-Cl activates benzene and catalyzes the exchange of deuterium between arene CH bonds and especially in the presence of solvent donor X-D bonds, where X is the conjugate base of an acid solvent, or the C-D bond of a donor hydrocarbon. Thus when benzene and toluene-d₃ are heated to 170° C. with or with added 1-Cl, little H/D exchange is observed (entries 1-3 in Table 1). In contrast, when 1-Cl (0.24 mg, 3.07×10−³ mmol) was heated at 170° C. in C₆H₆/DOAc (0.2 ml/11 ml) for 30 min, (Entry 6 in Table 1) 53% of the C₆H₆ is consumed (TON=5720, TOF=3.18 s−¹). As shown in Table 1, even acetic acid (a weak organic acid) enhances the activity of H/D exchange.

TABLE 1
H/D exchange between benzene^{a} and DX.

| | | Conc. | Time | | | Benzene isotopologues | | | | | | |
|---|---|---|---|---|---|---|---|---|---|---|---|---|
| entry | Catalyst | (mol %) | (h) | | DX | H₆ | D₁ | D₂ | D₃ | D₄ | D₅ | D₆ |
| 1 | 1-Cl^{b} | 0.04 | 1 | | C₆H₅CD₃ 1 ml | 96.28 | 3.67 | 0.04 | 0 | 0 | 0 | 0 |
| 2 | | | 6.7 | | C₆H₅CD₃ 1 ml | 95.36 | 4.5 | 0.1 | 0.02 | 0.01 | 0.01 | 0 |
| 3 | 1-Cl^{b} | 0.02 | 1 | | C₆H₅CD₃ 1 ml | 97.23 | 2.76 | 0.01 | 0 | 0 | 0 | 0 |
| 4 | 1-Cl | 1.0 | 0.5 | | DOAc 1 ml | 0.24 | 4.84 | 19.14 | 31.42 | 28.44 | 13.25 | 2.57 |
| 5 | | | 1 | | | 0.0 | 2.87 | 14.57 | 29.6 | 31.61 | 17.56 | 3.79 |
| 6 | 1-Cl | 1.0 | 0.5 | | DOAc 1 ml | 0.39 | 4.02 | 16.16 | 30.25 | 30.07 | 15.83 | 3.27 |
| 7 | | | +0.5 | | +1 ml DOAc | 0 | 0 | 2.36 | 14.58 | 33.09 | 34.80 | 15.18 |
| 8 | 1-Cl | 1.0 | 1 | | DOAc 2 ml | 0 | 0 | 2.72 | 14.43 | 32.64 | 35.15 | 15.06 |
| 9 | 1-Cl | 1.0 | 0.5 | | DOAc 1 ml | 0 | 0 | 0 | 0.94 | 12.56 | 40.97 | 45.52 |
| | | | | | D₂O 1 ml | | | | | | | |
| 10 | 1-Cl | 0.016 | 0.5 | | DOAc 1 ml | 47.03 | 28.13 | 15.25 | 6.62 | 2.35 | 0.56 | 0.06 |
| 11 | 1-Cl | 0.017 | 0.25 | | DOAc 1 ml | 75.97 | 15.50 | 6.07 | 1.89 | 0.47 | 0.09 | 0.01 |
| 12 | 1-Cl:Hg | 0.017 | 0.25 | | DOAc 1 ml | 71.97 | 17.83 | 7.19 | 2.28 | 0.61 | 0.11 | 0.01 |
| 13 | 1-Cl^{c} | 1.0 | 3 | days | DOAc 2 ml | 19.58 | 20.99 | 21.50 | 17.65 | 12.17 | 6.33 | 1.79 |
| 14 | | | 6 | day | | 4.79 | 12.41 | 19.43 | 23.84 | 21.91 | 13.51 | 4.11 |

^{a}Reaction conditions: 170° C., argon, [C₆H₆] = 2.24 mmol, 0.2 ml.
^{b}Reaction conditions: 170° C., argon, [C₆H₆] = 5.60 mmol, 0.5 ml.
^{c}Reaction conditions: 60° C., argon, [C₆H₆] = 2.24 mmol, 0.2 ml.

1-Cl activates the aromatic CH bond of benzene and readily catalyzes H/D exchange deuterated acetic acid as well as toluene. Thus, when 1 Cl (1 mol %) was heated at 170° C. in C₆H₆/DOAc for 30 min, >99% of the C₆H₆ was converted to a mixture of benzene isotopologs (4.8% D₁, 19.1% D₂, 31.5% D₃, 28.4% D₄, 13.3% D₅, 2.6% D₆) (Table 1, entry 4). Consistent with this high activity, more exchange is observed at longer time (Entry 5) and the system reaches equilibrium in <6 h. Confirming the high stability of the system, additional acetic acid-d₁ leads to further catalysis and exchange (entries 6 and 7). No exchange between C₆H₆ and DOAc is observed in the absence of added 1-Cl.

Consistent with the possible formation of less active, Cl-bridged dinuclear complexes, higher catalytic rates are observed at lower concentrations of 1-Cl (0.016 mol % entry 10) and after 30 min 53% of the C₆H₆ is consumed (TON=5720, TOF=3.18 s−¹). This is faster than the previously reported (acac-O,O)₂IrMeL system (TOF˜2 s−¹ at 180° C.). H/D exchange can thus be performed at lower temperatures, and 1-Cl is longer lived and still active after 6 days at 60° C.

Addition of H₂O does not inhibit the reaction of 1-Cl, consistent with 1-Cl as a distinct new species of alkane activation catalysts. As previously mentioned, catalysts based on Pt(II) and Hg(II) are inhibited by water. In contrast, when a solution of catalyst 1-Cl (14.1 mg 0.0224 mmol) in the presence of added water (D₂O) was heated at 170° C. in C₆H₆/DOAc/D₂O (0.2 ml/1 ml/1 ml) for 30 min (Entry 9 in Table 1), there was no decrease in product formation (TON=434 with out water, TON=530 with water), in fact a greater TON was observed due to the higher molar concentration of deuterium available for exchange. In a control experiment to rule out the possible role of iridium metal, addition of elemental mercury (Hg: Ir; 3000:1) showed no decrease in rate or activity as would be expected if the catalyst was iridium metal (Entry 12).

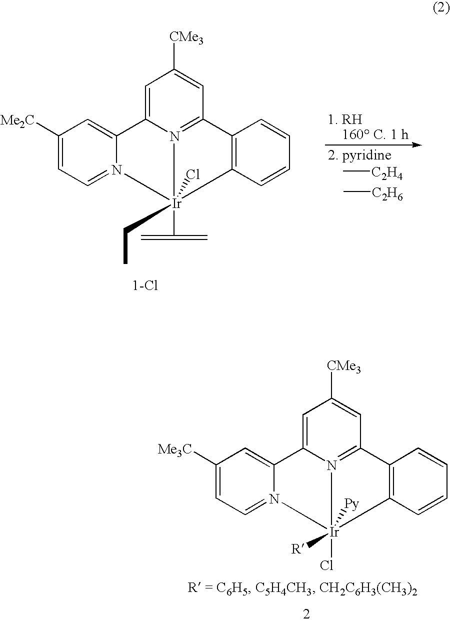

Further evidence that the catalyst systems are distinct and operate by CH activation, the stoichiometric CH activation of benzene in the non-acidic solvents was investigated. Heating 1-Cl in neat C₆H₆ at 160° C., (Eq. 2), produced a dinuclear complex [Ir(NNC)Cl(C₆H₅)]₂, [2]₂, characterized on the basis of NMR analysis. This product is a dimeric form of an initial CH activation of benzene. The synthesis and characterization of [2]₂ is described in Example 3 below.

Treatment of the reaction mixture containing [2]₂ with pyridine allowed the isolation of Ir(NNC)Cl(Py)(C₆H₅) (2-Py) in 92.5% yield (Example 4 below). Complex 2-Py has been characterized by NMR, elemental analysis, and x-ray crystallography.

CH activation can also be carried out under air (with or without added H₂O), and 2-Py can be obtained in good yields (˜74%). Treating 1-Cl with toluene showed only aromatic CH activation (p:m:o ratio of 3:5:1), while preliminary results with mesitylene indicate benzylic CH activation also occurs.

### Benzene Activation-Theoretical

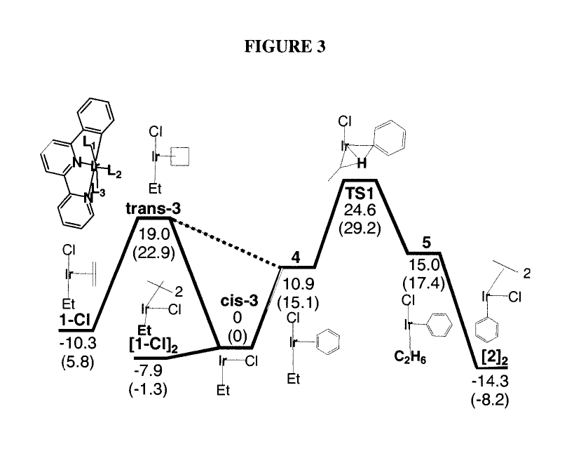

DFT calculations (B3LYP/LACVP** with ZPE and implicit benzene solvent corrections) support a mechanism summarized in FIG. 3. Reaction proceeds by loss of C₂H₄ from 1-Cl, initially generating a 5-coordinate species, trans-3 that isomerizes to a more stable 5-coordinate, ground state species, cis-3, that is in equilibrium with the dinuclear complex [Ir(NNC)EtCl]₂, [1-Cl]₂. Consistent with calculations, [1-Cl]₂ shows the same rate of reaction (k˜2.4×10−⁴ s−¹, 120° C.) as 1-Cl.

From cis-3, a transition state for CH activation was found (TS1), with a ΔG^{‡}=29.2 kcal/mol. This transition state (FIG. 3) is characterized by a fully formed bond between the hydride and the metal, reminiscent of our previously described OHM TS that can be considered a concerted combination of oxidative addition and reductive elimination steps. Consistent with the calculated ΔH, the rate at which [1-Cl]₂ disappears when heated in neat C₆H₆ between 100 and 140° C. lead to a ΔH^{‡}=26.8±3.7 kcal/mol, ΔS^{‡}≈−7.3 cal/mol.K. From TS1, the system loses ethane and forms [2]₂ (or, upon addition of pyridine, 2-Py) with ΔG=−8.2 kcalμmol. This is consistent with the experimental observation that the phenyl complex [2]₂, is generated in good yields as described in Example 2 below.

Computational studies with methane proved promising, and when 1-Cl (0.4 mol %) was heated in trifluoroacetic acid-dl solvent at 180° C. for 3.5 h, ˜2.4% of the methane was converted to deuterated isotopologs, mainly consisting of CH₃D (2.1%, TON=6.2). Significantly, under identical conditions, the corresponding (NNC)Pt(II) or Pt(bpym)Cl₂ complexes showed no reaction. While the solution of 1-Cl remained homogeneous, the catalytic activity slowly dropped with time (likely due to formation of inactive dinuclear, Cl-bridged complexes), and after ˜21 h a total of 12 turnovers was observed. No significant H/D exchange was observed with 1-Cl in D₂SO₄, although trace amounts of methanol were detected.

In order to prevent formation of the chloro-bridged, dinuclear species, [1-Cl]₂ that was likely inhibiting reaction in acid solvent, the Cl⁻ ligand was replaced with the less coordinating CF₃CO₂⁻ group. Treatment of 1-Cl with AgTFA in CH₂Cl₂ precipitated AgCl and generated a new soluble complex tentatively identified as Ir(NNC)(Et)(TFA)(C₂H₄), 1-TFA.

Another embodiment of the present invention are catalyst systems capable of activating the CH bonds of alkanes such as methane. The activation step can be detected using H/D exchange reaction between methane and trifluoroacetic acid-d₁, at temperatures between 100 and 180° C. H/D exchange indicated of CH activation and was useful for screening catalysts without having to involve other catalyst components necessary for a complete catalytic cycle. Typically, H/D exchange reactions were performed in a deuterated solvent system, and deuterium incorporation into alkane or arene was probed.

A solution of 1-TFA (0.3 mol %) in trifluoroacetic acid-d₁, efficiently catalyzed the H/D exchange between methane and trifluoroacetic acid-d₁. After 2 h at 180° C., ˜30% of the methane was converted to all four deuterated isotopologs (TON=153, TOF=0.021 s⁻¹). The rate of CH activation of methane in TFA approached that for Pt(bpym)Cl₂ (which is inactive in TFA) in strong sulfuric acid. Consistent with the Pt(bypm) and Hg(II) systems behaving as an electrophilic catalyst, that are inhibited by weaker acids, no HID exchange reaction between methane and trifluoroacetic acid-d₁ were observed with these systems. Thus, when Pt(bpym)TFA₂ (7.7 mg, 0.0134 mmol) was heated at 180° C. with methane (500 psi, 5 mmol) in trifluoroacetic acid-d1 (1 ml) for 2 h, no deuterium incorporation into methane was observed. Similarly, no reaction was observed with Hg(TFA)₂ in CF₃CO₂H.

Further examples of CH activation of methane showed that solutions of Ir(NNC)Et(TFA)(C₂H₄), 1-TFA (9.5 mg, 0.0134 mmol) in trifluoroacetic acid-d1 (1 ml), efficiently catalyzed the H/D exchange between methane (500 psi, 5 mmol) and trifluoroacetic acid-d₁. After 2 h at 180° C.,˜30% of the methane was converted to all four deuterated isotopologs (20.7% CH3D, 6.1% CH₂D₂, 1.2% CHD₃, 0.6% CD₄; TON=153, TOF=0.021 s⁻¹).

The temperature dependence (105-135° C.) on the rate of H/D exchange between methane and trifluoroacetic acid-d¹ with 1-TFA, allowed the activation barrier for CH activation to be determined. Consistent with the high activity of this system, an activation barrier of ΔH^{|=}21.7±2.4 kcal/mol was obtained. It is significant that this systems is faster in the weaker acid, CF₃CO₂H (ΔH^{|=}21.7±2.4 kcal/mol) than the Pt(bpym)Cl₂ is in the stronger acid, H₂SO₄ (ΔH^{|=}28.0±2.4 kcalμmol), confirming that more active catalysts can be developed with more electron-rich system, that are thermally stable to acidic, oxidizing conditions, and operate in a manner distinct from the Hg and Pt systems.

Treatment of 1-TFA with TFA leads to the release of ethane and likely in-situ formation of the bis trifluoroacetate complex, which has been partially characterized. Given the high thermal and protic stability of these Ir(NNC)complexes, the complexes were tested for the conversion of methane to methanol or methyl esters in weaker acids such as TFA. When a solution of 1-TFA in TFA was heated with methane at 180° C. for 3 h with NaIO₄ as an oxidant, methyl trifluoroacetate was produced with a TON=6.3.

Embodiments of the present invention demonstrated that CH activation proceeded under very mild conditions with high selectivity. Coupling such a CH activation reaction to a functionalization reaction whereby a M-R intermediate is converted to a useful, functionalized product such as an alcohol or ester, along with regeneration of the catalyst, MX, leads to an effective catalytic cycle for the selective conversion of hydrocarbons to functionalized products.

Given the thermal and acidic stability of Ir(NNC) complexes disclosed herein, and their high activity for CH activation, the complexes were tested for the selective oxidation of alkanes.

The conversion of methane to functionalized product requires more than CH activation. A successful conversion of alkanes to esters in acids such as TFA was carried out in the presence of functionalization reagents that could functionalize the Ir—CH₃ intermediates to an oxygenated product.

When a 14.87 mM solution of Ir(NNC)Et(TFA)(C₂H₄) (10.5 mg, 0.0149 mmol) in trifluoroacetic acid-d1 (1 ml) was heated with methane (500 psi, 5 mmol) at 180° C. for 3 h with NaIO₄ (90 mg, 0.421 mmol) as the oxidant, 6.3 turnovers of methyl trifluoroacetate (0.0936 mmol) were produced. The formation of methyl trifluoroacetate was verified by 1H, ¹³C, and ¹⁹F NMR, GC-MS, and HPLC. To verify that the methyl trifluoroacetate observed was generated from the activation and functionalization of methane, the reaction was carried out with 13C labeled methane. These labeling studies verified that the CH₃TFA produced was from methane and that 13CH₃TFA was observed by GC-MS and ¹³C NMR. Thus, when a 21.91 mM solution of Ir(NNC)Et(TFA)(C₂H₄) (17.3 mg, 0.0219 mmol) in trifluoroacetic acid-d1 (1 ml) was heated with methane (99% minimum ¹³C enriched, 400 psi, 4 mmol) at 180° C. for 3 h with NaIO₄ (79.6 mg, 0.3722 mmol), 13CH₃TFA was observed by GC-MS and ¹³C NMR.

The Ir(NNC) system, 1-TFA activated other linear and cyclic alkanes, such as octane, cyclooctane, and cyclohexane in trifluoroacetic acid as evidenced by incorporation of deuterium into the alkane. The rate and extent of incorporation was followed by GC-MS. Control reactions without catalyst were performed, lacked observable isotope exchange between alkane and trifluoroacetic acid.

Embodiments of the present invention are further illustrated by the non-limiting examples.

General Considerations: Unless otherwise indicated, all reactions and manipulations were performed using standard Schlenk techniques under argon or in an MBraun LABmaster 130 glove box under nitrogen. Ultra high purity argon was used as is after passing through a column of Drierite to remove residual moisture.

For DFT calculations: All calculations were carried out using B3LYP/LACVP** as implemented by the Jaguar 6.0 and Jaguar 6.5 program packages (Jaguar 6.0, Schrodinger, LLC, Portland, Oreg., 2005). Other supporting information is found in: Becke, A. D. J. Chem. Phys. 1993, 98, 5648; Lee et al. Phys. Rev. B 1988, 37, 785; Hay et al. J. Chem. Phys. 1985, 82, 299; Goddard et al. Phys. Rev. 1968, 174, 659; Melius et al., Chem. Phys. Lett. 1974, 28, 457.

All reported energies are solvent corrected [using the Poisson-Boltzman continuum solvent method (ε=2.284 and solvent radius=2.60219 Å)] enthalpies at 0 K (including ZPE).

IrCl₃ (Pressure Chemical), phenyl lithium (1.8M in butyl ether, Aldrich) were used as received. All solvents were reagent grade or better. Ether, benzene, pentane, and dichloromethane were dried and deoxygenated by sparging with argon and then passing through activated alumina using an mBraun MB-SPS solvent purifier system.

### Analysis of Reaction Products:

Reaction products were analyzed by GC-MS, HPLC, and NMR. ¹H (400 MHz), ³C (100 MHz), and ¹⁹F NMR (376.5 MHz) spectra were collected on a Varian 400 Mercury plus spectrometer. Chemical shifts were referenced using residual solvent proton signal. All coupling constants are reported in Hz. Chemical shifts were assigned based on g-COSY, g-HSQC, and g-MHBC or cigar experiments. Mass spectrometric analyses were performed at UCLA and UCR Mass Spectrometry Facility. Elemental analysis were performed by Desert Analytical Laboratory, Inc.; Arizona.

The extent of H/D exchange of the methane gas was determined by GC using a Shimadzu GC-MS QP5000 (ver. 2) equipped with a GS-gaspro column. The liquid phase was analyzed by ¹H, ¹³C, and ¹⁹F NMR, which supported that methyl trifluoroacetate, was formed, and had identical chemical shifts when compared to authentic samples. The amount of methyl trifluoroacetate produced was quantified by acetic acid as an internal reference. In the case of methyl trifluoroacetate, TON (turnover number) is defined as (moles of methyl trifluoroacetate)/(moles of catalyst) and the TOF (turnover frequency) is defined as TON/time (s⁻¹)

The products were also verified by GC-MS with a Shimadzu GC-MS QP5000 (ver. 2) equipped with a cross-linked methyl silicone gum capillary column (DB5), and by HPLC with a Varian Pro Star HPLC equipped with a Aminex© HPX87H Organic acid analysis column and a refractive index detector, and match authentic samples. The eluant was 0.01% H₂SO₄ in water.

NNC ligand, 6-phenyl-4,4′-di-tert-butyl-2,2′-bipyridine was prepared following literature procedure of Lu et al. (J. Am. Chem. Soc, 2004. 126, 4958).

### Example 1

### Syntheses of 1-Cl and 1-Cl—NCCH₃

[Ir(C₂H₄)₂Cl]₂ (1.03 g, 1.82 mmol) was dissolved in CH₂Cl₂ (25 ml) in a thick walled glass vacuum bulb equipped with a PTFE valve. In a separate vessel, NNC ligand 6-phenyl-4,4′-di-tert-butyl-2,2′-bipyridine (1.25 g, 3.63 mmol) was dissolved in CH₂Cl₂ (15 ml). Ethylene was then bubbled through the iridium-containing solution while stirring at −50° C. for 5 min. Solution containing the dissolved ligand was then cannula-transferred into the iridium containing solution. The flask containing the ligand was then washed with CH₂Cl₂ (15 ml) and transferred over to ensure complete transfer. The red solution was then stirred at −50° C. for 15 min, then warmed to room temperature and stirred for 16 h. During the course of formation of 1-Cl, the reaction vessel was opened periodically to vent excess ethylene pressure. The solvent was then reduced to 20 ml under reduced pressure, and ethylene was then bubbled through for 5 min.

1-Cl can be converted to 1-Cl—NCCH₃ by ethylene displacement by nitrile. In one embodiment, excess acetonitrile (20 ml) was added to a solution of 1-Cl prepared as above and the solution was heated at 50° C. for 30 min. The solvent was then removed, and the resulting red residue was passed through neutral alumina with CH₂Cl₂ until the yellow band came off, then ethyl acetate/methanol gradient to remove 1-Cl (orange band) and acetonitrile complex 1-Cl—NCCH₃ (red band). Complexes 1-Cl and 1-Cl—NCCH₃ were obtained as crystalline materials by recrystallization from CH₂Cl₂/pentane at −25° C. Yield: 1.32 g (57.9%) of 1-Cl and 301.5 mg (13%) of 1-Cl—NCCH₃.

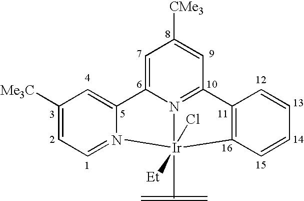

¹H NMR of 1-Cl: (CDCl₃) 9.21 (d, 1H, ³J=6.1, H-1), 8.04 (d, 1H, ⁴J=2.0; H-4), 7.94 (d, 1H, ⁴J=1.6, H-7), 7.80 (d, 1H, ⁴J=1.6, H-9), 7.73 (dd, 1H, ³J=8.0 ⁴J=1.0, H-1S), 7.68 (dd, 1H, ³J=7.8 ⁴J=1.6, H-12), 7.56 (dd, 1H, ³J=6.1 ⁴J=2.1, H-2), 7.28 (dt, 1H, ³J=7.5 ⁴J=1.6, H-14), 7.13 (dt, 1H, ³J=7.5 ⁴J=1.0, H-13), 3.97 (s, 4H, C₂H₄), 1.52 (s, 9H, CMe₃), 1.48 (s, 9H, CMe₃), 0.47 (dq, 1H, 2j=10.8 ³J=7.7, —CH₂—), 0.25 (dq, 1H, 2j=10.8 ³J=7.7, —CH₂—), −0.28 (t, 3H, ³J=7.7, —CH₃).

¹³C{¹H}NMR of 1-Cl (CDCl₃): 163.88 (C-8), 163.04 (C-3), 162.28 (C-16), 158.40 (C-6), 153.18 (C-5), 151.21 (C-1), 144.76 (C-10), 144.38 (C-11), 134.99 (C-15), 131.67 (C-13), 124.90 (C-12), 124.63 (C-2), 122.74 (C-14), 119.61 (C-4), 116.17 (C-7), 115.13 (C-9), 65.86 (C₂H₄), 35.59 (CMe₃), 35.47 (CMe₃), 30.94 (CMe₃), 30.62 (CMe₃), 14.95 (—CH₃), −7.52 (—CH₂—).

ESI-MS: 593.2 (M−Cl)⁺, 565.2 (M−Cl —C₂H₄)⁺.

Elemental analysis; Found: C, 52.98; H, 5.52; N, 4.24; Cl 5.59 Calculated; C, 53.53; H, 5.78; N, 4.46; Cl, 5.64

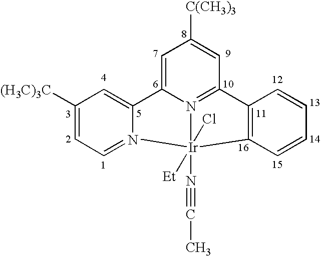

¹H NMR of 1-Cl—NCCH₃ (CDCl₃) 8.78 (d, 1H, ³J=5.7, H-1), 7.91 (d, 1H, ⁴J=1.8, H-4), 7.66 (d, 1H, 4j=1.6, H-7), 7.61 (d, 1H, ⁴J=1.6, H-9), 7.55 (m, 2H, H-12, 15), 7.48 (dd, 1H, ³J=5.7 ⁴J=1.8, H-2), 7.14 (dt, 1H, ³J=7.7, 7.3, ⁴J=1.4, H-13), 6.98 (dt, 1H, ³J=7.6, 7.4, ⁴J=1.3, H-14), 2.69 (s, 3H, NCCH₃), 1.44 (s, 9H, CMe₃), 1.43 (s, 9H, CMe₃), 0.91 (m, 1H, —CH₂—), 0.67 (m, 1H, —CH₂—), 0.21 (t, 1H, ³J=7.7, —CH₃).

¹³C NMR of 1-NCCH₃ (CDCl₃) 167.79 (C-16), 161.83 (C-8), 160.88 (C-3), 157.64 (C-6), 155.59 (C-5), 154.08 (C-10), 149.98 (C-1), 145.21 (C-11), 133.67 (C-15), 130.99 (C-13), 124.90 (C-12), 124.12 (C-2), 120.75 (C-14), 119.32 (C-4), 115.07 (NCCH₃), 114.82 (C-7), 114.06 (C-9), 35.38 (CMe₃), 31.07 (CMe₃), 30.74 (CMe₃), 16.05 (—CH₃), 5.07 (NCCH₃), −9.47 (—CH₂—). ESI-MS: 647.2 (M-Cl +NCCH₃)+606.2 (M-Cl)⁺, 565.2 (M-Cl—NCCH₃)⁺. Elemental analysis: Found: C, 52.03; H, 5.47; N, 6.23; Cl, 5.62 Calculated; C, 52.44; H, 5.50; N, 6.55; Cl, 5.53.

### Example 2

### Synthesis of [Ir(NNC)EtCl]2, [1-Cl]2

A thick-walled glass vacuum bulb equipped with a PTFE valve was loaded with 1-Cl (70 mg, 0.111 mmol) and dioxane (6 ml). The orange solution was heated at 100° C. for 2 h. The solvent was then removed under vacuum and the resulting green residue was then passed through alumina with CH₂Cl₂ then THF. Complex [1-Cl]₂ was then recrystallized from CH₂Cl₂/pentane at −20° C. ¹H NMR (CDCl₃) 8.81 (d, 1H, ³J=5.8, H-1), 7.92 (d, 1H, 4j=1.5, H-4), 7.63 (s, 1H, H-7), 7.62 (s, 1H, H-9), 7.59 (d, 1H, ³J=7.8, H-15), 7.40 (dd, 1H, ³J=5.7, ⁴J=1.9, H-2), 7.36 (d, 1H, ³J=7.4, H-12), 7.05 (dt, 1H, ³J=7.4, ⁴J=1.3, H-14), 6.92 (dt, 1H, ³J=7.4, ⁴J=1.3, H-13), 1.53 (s, 9H, CMe₃), 1.47 (s, 9H, CMe₃), 0.64 (m, 1H, —CH₂—), 0.48 (m, 1H, —CH₂—), −0.36 (t, 1H, ³J=7.6, —CH₃). ¹³C NMR (CDCl₃) 169.99, 161.01, 159.61, 157.71, 157.46, 157.23, 152.43, 147.1, 137.47, 130.51, 124.57, 124.31, 120.19, 118.18, 113.86, 113.72, 35.49, 35.32, 31.21, 30.98, 15.39, −8.19.

### Example 3

### Synthesis of [Ir(NNC)Cl(C₆H₅)]_{2[}2]₂

Complex 1-Cl (100 mg, 0.159 mmol) was heated at 160° C. in benzene (100 ml) in a thick-walled glass vacuum bulb equipped with a PTFE valve for 2 h. The solvent was then removed under vacuum, the residue was redissolved in CH₂Cl₂, and reprecipitated with pentane.

Yield 94.7 mg (91.8%). Elemental analysis: Calculated: C, 55.58; H, 4.98; N, 4.32; Cl 5.47 Found: C, 55.19; H, 4.69; N, 4.76; Cl, 5.20.

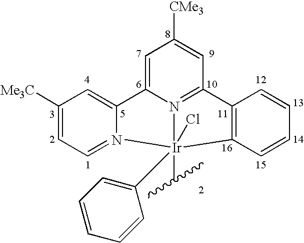

¹H NMR (CD₂Cl₂) 8.57 (d, 1H, ³J=5.8, H-1), 8.05 (d, 1H, ⁴J=1.7, H-4), 7.80 (bs, 2H, H-7, 9), 7.69 (dd, 1H, ³J=7.9 ⁴J=1.3, H-15), 7.43 (dd, 1H, ³J=5.8, ⁴J=1.7, H-2), 7.15 (dd, 1H, ³J=7.4 ⁴J=1.3, H-12), 7.10 (dt, 1H, ³J=7.5 ⁴J=1.3, H-14), 6.91 (dt, 1H, ³J=7.44J=1.3, H-13), 6.40-6.28 (m, 5H, phenyl), 1.53 (s, 9H, CMe₃), 1.52 (s, 9H, CMe₃). ¹³C NMR (CD₂Cl₂) 169.20 (C-16), 162.27 (C-3), 161.89 (C-8), 157.41 (C-5), 157.08 (C-6), 154.36 (C-10), 152.14 (C-1), 147.66 (C-11), 137.39 (C-15), 133.73 (Phenyl), 130.92 (C-14), 125.31 (Phenyl), 125.11, 124.75, 124.70, 121.35, 119.35 (C-13), 115.25 (C-7), 115.20 (C-9), 35.65 (CMe₃), 31.00 (CMe₃), 30.69 (CMe₃).

When an analogous reaction starting with 1-Cl—NCCH₃ was carried out in C₆D₆, multiple deuterated ethane isotopologs (major product) and deuterated ethylene (minor product) were observed by GC-MS, suggesting that beta hydride elimination is occurring, and is fast and reversible. Consistent with a hydride intermediate, ¹H NMR analysis of [2]2 indicates that the ortho proton (H-12) of the back of the phenyl ring of the ligand has become partially deuterated (˜40%).

### Example 4

### Synthesis of Ir(NNC)Cl(C₆H₅)Py(2-Py)

Complex 1-Cl (52 mg, 8.28×10⁻² mmol) was heated at 160° C. in benzene (80 ml) in a thick-walled glass vacuum bulb equipped with a PTFE valve for 1 h. The solvent was removed under vacuum, and then redissolved in pyridine (10 ml). Solvent was removed and the residue was purified by alumina prep TLC using CH₂Cl₂. Pure 2-Py was precipitated from CH₂Cl₂/pentane. Yield 55.7 mg (92.5%).

ESI-MS: 750.2 (M+Na)⁺ 728.2 (M+H)⁺.

Elemental analysis for 2-Py: •Found C, 56.97; H, 4.86; Cl, 4.87; N, 5.74. Calc. C, 57.79; H, 5.13; Cl, 4.87; N, 5.78.

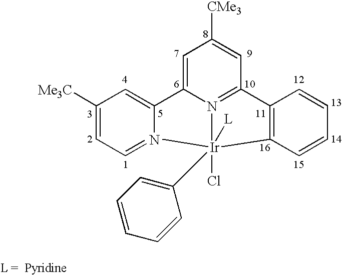

¹H NMR (CDCl₃) 9.30 (d, 1H, ³J=5.8, H-1), 8.32 (dd, 2H, o-Py), 8.07 (dd, 1H, ³J=7.6 ⁴J=1.2, H-15), 7.87 (d, 1H, ⁴J=1.7, H-4), 7.72 (d, 1H, ³J=1.8, H-7), 7.63 (dd, 1H, ³J=5.7 ⁴J=1.9, H-2), 7.61 (d, 1H, ³J=1.7, H-9), 7.56 (dd, 1H, ³J=7.8, H-12), 7.47 (m, 1H, p-Py), 7.19 (dt, 1H, ³J=7.6 ³J=1.3, H-14), 7.04 (dt, 1H, ³J=7.6 ³J=1.2, H-13), 6.97 (m, 2H, m-Py), 6.88 (m, 2H, o-Phenyl), 6.62-6.54 (m, 3H, m-phenyl, p-phenyl), 1.47 (s, 9H, CMe₃), 1.44 (s, 9H, CMe₃). ¹³C NMR (CDCl₃) 169.50, 162.43, 160.50, 158.24, 156.50, 156.46, 150.54, 149.57, 146.02, 136.51, 136.35, 135.79, 131.85, 127.70, 125.42, 124.92, 124.85, 121.52, 121.20, 119.18, 115.18, 114.62, 35.57, 35.52, 31.14, 30.81.

### Example 5

### Synthesis of Ir(NNC)TFA(Et)(C₂H₄) (1-TFA)

Complex 1-Cl (376 mg, 0.599 mmol) and silver trifluoroacetate (169 mg, 0.659 mmol) were stirred at room temperature in CH₂Cl₂ (15 ml) in the dark for 1 day. The resulting suspension was filtered over celite, and the filtrate was collected. Orange-yellow microcrystalline material was obtained from a CH₂Cl₂/pentane solution at −25° C.

Yield: 427.7 mg (96.2%).

¹H NMR (CDCl₃) 9.24 (d, 1H, ³J=6.2, H-1), 8.04 (d, 1H, ⁴J=2.1, H-4), 7.94 (d, 1H, ⁴J=1.6, H-7), 7.81 (d, 1H, ⁴J=1.6, H-9), 7.71 (d, 1H, ³J=8.0, H-1S), 7.68 (dd, 1H, ³J=7.7 ⁴J=1.6, H-12), 7.62 (dd, 1H, ³J=6.1 ⁴J=2.1, H-2), 7.32 (dt, 1H, ³J=7.8, 7.5, H-14), 7.19 (t, 1H, ³J=7.7, 7.4, H-13), 4.04 (m, 2H, C₂H₄), 3.95 (m, 2H, C₂H₄), 1.54 (s, 9H, CMe₃), 1.50 (s, 9H, CMe₃), 0.8 (m, 1H, ³J=10.5, 7.7, —CH₂—), 0.54 (dq, 1H, ³J=10.5, 7.7, —CH₂—), −0.47 (t, 3H, ³J=7.7, —CH₃).

¹³C NMR (CDCl₃) 164.1, 164.36, 164.12, 159.53, 154.86, 151.32, 145.93, 135.57, 132.85, 131.75, 125.12, 124.79, 124.77, 120.06, 116.70, 116.07, 35.69, 35.58, 30.92, 30.58. ¹⁹F NMR (CDCl₃) −78.99.

Elemental analysis: Found C, 50.75; H, 4.98; F 8.14; N, 3.96 Calc. C, 51.05; H, 5.14; F, 8.07; N, 3.97.

### Example 6

### Reaction of 1-Cl with Toluene

In a thick-walled glass vacuum bulb equipped with a PTFE valve. 1-Cl (125 mg, 0.199 mmol) was dissolved in toluene (150 ml), and heated at 170° C. for 5.5 h. The solvent was then removed under vacuum. The resulting red residue was then dissolved in pyridine and heated at 70° C. for 45 min. The solvent was then removed under vacuum. ¹H NMR showed a 5.5:4.8:1 ratio of isomers (based on toluene CH₃ group). The toluene activation product was then obtained by separation on a silica preparative TLC plate with CH₂Cl₂ then recrystallized from CH₂Cl₂/ether. Elemental analysis: Found C, 58.31; H, 5.17; N, 5.28; Calc. C, 58.32; H, 5.30; N, 5.67. meta-isomer ¹H NMR (CDCl₃) 9.29 (d, 1H ³J=5.5, H-1), 8.30 (d, 2H, o-Py), 8.05 (d, 1H, 3j=7.4, H-1S), 7.85 (d, 1H, ⁴J=1.7, H4), 7.71 (d, 1H, ⁴J=1.7, H7), 7.61 (dd, 1H, ³J=5.4, ⁴J=1.7, H2), 7.59 (dd, ⁴J=1.7, H9), 7.55 (dd, ³J=7.7, ⁴J=0.8, H12), 7.46 (t, 1H, p-Py), 7.17 (dt, 1H, ³J=7.4, ⁴J=1.4, H14), 7.03 (dt, 1H, ³J=7.5, ⁴J=1.4, H13), 6.96 (t, 1H, m-Py), 6.82 (s, 1H, o-tolyl), 6.51 (d, 1H, p-tolyl), 6.44 (t, 1H, m-tolyl), 6.37 (d, 1H, o-tolyl), 1.96 (s, 3H, tolyl-CH₃), 1.45 (s, 9H, CMe₃), 1.42 (s, 9H, CMe₃), para-isomer ¹H NMR (CDCl₃) 9.28 (d, 1H ³J=5.5, H-1), 8.30 (d, 2H, o-Py), 8.05 (d, 1H, ³J=7.4, H-15), 7.86 (d, 1H, ⁴J=1.7, H-4), 7.70 (d, 1H, ⁴J=1.7, H-7), 7.61 (dd, 1H, ³J=5.4, ⁴J=1.7, H-2), 7.60 (dd, ⁴J=1.7, H-9), 7.53 (dd, ³J=7.7, ⁴J=0.8, H-12), 7.46 (t, 1H, p-Py), 7.17 (dt, 1H, ³J=7.4, ⁴J=1.4, H-14), 7.02 (dt, 1H, ³J=7.5, ⁴J=1.4, H-13), 6.96 (t, 1H, m-Py), 6.74 (d, 2H, m-tolyl), 6.42 (d, 2H, o-tolyl), 2.00 (s, 3H, tolyl-CH₃), 1.45 (s, 9H, CMe₃), 1.42 (s, 9H, CMe₃).

### Example 7

### Reaction of 1-Cl with Mesitylene

In a thick-walled glass vacuum bulb equipped with a PTFE valve, 1-Cl (53 mg, 0.0844 mmol) was dissolved in mesitylene (50 ml) and heated at 190° C. for 19 h. Pyridine was then added to the solution and the solvent was then removed under vacuum. ¹H NMR of the resulting residue showed that the CH₃ group has been activated, forming two isomers.

### Example 8

### H/D Exchange Between Benzene and Acetic Acid-d₁ Catalyzed by 1-Cl

H/D exchange reactions between benzene and acetic acid-d₁ catalyzed by iridium complex 1-Cl were carried out in a 5 ml thick-walled glass vacuum bulb equipped with a PTFE valve. The extent of H/D exchange was determined by GC using a Shimadzu GC-MS QP5000 (ver. 2) equipped with a cross-linked methyl silicone gum capillary column (DB5). In the case of benzene, TON (turnover number) was defined as [(moles of benzene-d₁)+(moles of benzene-d₂)*2+(moles of benzene-d₃)*3 (moles of benzene-d₄)*4+(moles of benzene-d₅)*5+(moles of benzene-d₆)*6]/(moles of catalyst), where the molar amount of each isotopic species is derived from the table below. TOF (turnover frequency) is defined as TON/time (s⁻¹). Calculated results are collected in Table 1.

TABLE 1
H/D exchange between benzene^{a} and DX.

| | Conc. | Time | | Benzene isotopologues | | | | | | | |
|---|---|---|---|---|---|---|---|---|---|---|---|
| Catalyst | (mol %) | (h) | | DX | H₆ | D₁ | D₂ | D₃ | D₄ | D₅ | D₆ |
| 1-Cl^{b} | 0.04 | 1 | | C₆H₅CD₃ | 96.28 | 3.67 | 0.04 | 0 | 0 | 0 | 0 |
| | | | | 1 ml | | | | | | | |
| | | 6.7 | | C₆H₅CD₃ | 95.36 | 4.5 | 0.1 | 0.02 | 0.01 | 0.01 | 0 |
| | | | | 1 ml | | | | | | | |
| 1-Cl^{b} | 0.02 | 1 | | C₆H₅CD₃ | 97.23 | 2.76 | 0.01 | 0 | 0 | 0 | 0 |
| | | | | 1 ml | | | | | | | |
| 1-Cl | 1.0 | 0.5 | | DOAc | 0.24 | 4.84 | 19.14 | 31.42 | 28.44 | 13.25 | 2.57 |
| | | | | 1 ml | | | | | | | |
| | | 1 | | | 0.0 | 2.87 | 14.57 | 29.6 | 31.61 | 17.56 | 3.79 |
| 1-Cl | 1.0 | 0.5 | | DOAc | 0.39 | 4.02 | 16.16 | 30.25 | 30.07 | 15.83 | 3.27 |
| | | | | 1 ml | | | | | | | |
| | | +0.5 | | +1 ml | 0 | 0 | 2.36 | 14.58 | 33.09 | 34.80 | 15.18 |
| | | | | DOAc | | | | | | | |
| 1-Cl | 1.0 | 1 | | DOAc | 0 | 0 | 2.72 | 14.43 | 32.64 | 35.15 | 15.06 |
| | | | | 2 ml | | | | | | | |
| 1-Cl | 1.0 | 0.5 | | DOAc | 0 | 0 | 0 | 0.94 | 12.56 | 40.97 | 45.52 |
| | | | | 1 ml D₂O | | | | | | | |
| | | | | 1 ml | | | | | | | |
| 1-Cl | 0.016 | 0.5 | | DOAc | 47.03 | 28.13 | 15.25 | 6.62 | 2.35 | 0.56 | 0.06 |
| | | | | 1 ml | | | | | | | |
| 1-Cl | 0.017 | 0.25 | | DOAc | 75.97 | 15.50 | 6.07 | 1.89 | 0.47 | 0.09 | 0.01 |
| | | | | 1 ml | | | | | | | |
| 1-Cl:Hg | 0.017 | 0.25 | | DOAc | 71.97 | 17.83 | 7.19 | 2.28 | 0.61 | 0.11 | 0.01 |
| | | | | 1 ml | | | | | | | |
| 1-Cl^{c} | 1.0 | 3 | days | DOAc | 19.58 | 20.99 | 21.50 | 17.65 | 12.17 | 6.33 | 1.79 |
| | | | | 2 ml | | | | | | | |
| | | 6 | day | | 4.79 | 12.41 | 19.43 | 23.84 | 21.91 | 13.51 | 4.11 |

^{d}Reaction conditions: 170° C., argon, [C₆H₆] = 2.24 mmol, 0.2 ml.
^{e}Reaction conditions: 170° C., argon, [C₆H₆] = 5.60 mmol, 0.5 ml.
^{f}Reaction conditions: 60° C., argon, [C₆H₆] = 2.24 mmol, 0.2 ml.

### Example 9

### Measured Activation Energy for Benzene Activation

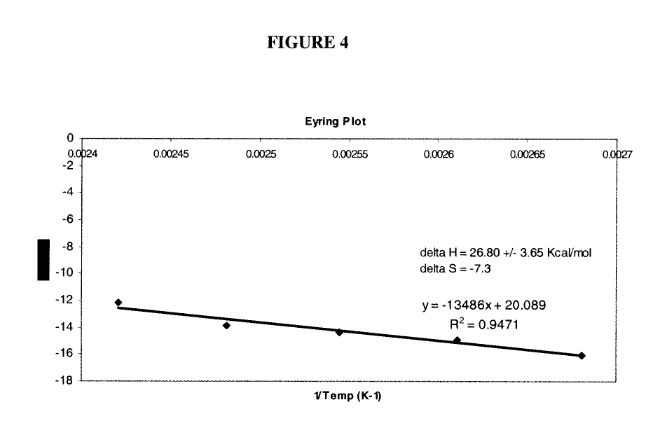

A 1.57 mM stock solution of 1-Cl in benzene was prepared, from which 10 ml aliquots were transferred to thick-walled glass vacuum bulbs equipped with a PTFE valves. The bulbs were then heated between 100 and 140° C. The reaction was monitored over time, by quickly cooling the reactor, then adding pyridine, and stirring for 30 min. The solvent was then removed and the residue was redissolved in CDCl₃ (0.8 ml) and the ratio of ethyl complex (1-C₁-Py) to phenyl product (2-Py) was determined. Values of ln[1-Cl-Py] was then plotted vs. 1/time to obtain k. The activation energy for the overall stoichiometric benzene activation was obtained from the Eyring plot shown in FIG. 4.

### H/D Exchange for Methane in Trifluoroacetic Acid-d1:

H/D exchange reactions between methane and trifluoroacetic acid-d1 (trifluoroacetic acid-d1) catalyzed by iridium complex 1-TFA were carried out in a stainless steal reactor with a glass insert equipped with a stir bar. The extent of HID exchange was determined by GC using a Shimadzu GC-MS QP5000 (ver. 2) equipped with a cross-linked methyl silicone gum capillary column (DB5). In the case of methane, TON (turnover number) is defined as [(moles of methane-d₁)+(moles of methane-d₂)*2+(moles of methane-d₃)*3 (moles of methane-d₄)*4]/(moles of catalyst) and the TOF (turnover frequency) is defined as TON/time (s⁻¹).

### Example 10

### H/D Exchange Catalyzed by 1-Cl and 1-TFA between Methane and Deuterated Acids

In a typical experiment a resealable metal reactor with a glass insert and stir bar was loaded with 10-20 mg of 1-Cl or 1-TFA, and then under argon 1 ml of solvent (trifluoroacetic acid-d1 or D₂SO₄) was added. The reactor was then flushed with 500 psi methane (3 times) and then pressurized with 500 psi methane while stirring. Control reactions were also prepared by identical procedures lacking catalyst. For the reactions with D₂SO₄, methane is known to undergo background HD exchange. The reactors was then placed in a preheated block at 180° C., and heated for 3 to 24 h. After reaction, the reactors were allowed to cool back to ambient temperature, and part of the headspace was transferred to a evacuate 2 ml vial fitted with a septa. The headspace of the catalyst runs and control runs were then analyzed by GC-MS and the control values were subtracted form the catalyst values to obtain corrected H/D exchange values. The reactors were then opened and the reaction mixtures were fully homogeneous with no signs of decomposition. For the reaction of 1-Cl with D₂SO₄, acetic acid (5 uL) was added to the reaction mixture and analyzed by ¹H NMR, which showed trace amounts of methanol (TON=0.04, 1%, where TON was calculated as moles of product/moles of catalyst). There was no background H/D exchange observed between methane and trifluoroacetic acid-d1. 1-Cl (0.39 mol %) was heated with methane (500 psi) in trifluoroacetic acid-d1 at 180° C. for 3.5 h, 2.1% CH₃D, TON=6.2, TOF=˜5.0×10⁴ sec⁻¹.

Catalytic H-D exchange reactions were quantified by monitoring the increase of deuterium into C₆H₆ by GC-MS analyses. Quantitative analysis was achieved by deconvolution of the mass fragmentation pattern obtained from the MS analysis, using a program developed with Microsoft EXCEL. An important assumption used in the program is that there are no isotope effects on the fragmentation pattern of the benzenes due to replacement of H with D. Fortunately, because of the relative stability of the parent ion towards fragmentation, it can be used reliably to quantify the exchange reactions. The mass range from 78 to 84 (for benzene) was examined for each reaction and compared to a control reaction where no metal catalyst was added. The program was calibrated with known mixtures of benzene isotopologues. The results obtained from this method are reliable to within 5%. Results are gathered in Table 1.

### Example 11

### H/D Exchange Catalyzed by 1-Cl in Neat Benzene and Toluene-d8

A stock solution of 1-10 mg of 1-Cl in 1.5 mL of (1:2 volume mixture of benzene and toluene-d₈. The solution were charged to 4 ml thick-walled glass bulbs equipped with a PTFE valve. The solutions were heated at 170° C. and followed over time by GC-MS. TON was calculated as (moles of D₁*1+moles of D₂*2+moles of D₃*3+ . . . )/moles of catalyst. Results are collected in Table B.

### Example 12

### H/D Exchange Catalyzed by 1-Cl between Benzene and Acetic Acid-d₄ Solvent

A stock solution of 1-10 mg/ml of 1-Cl in benzene were made. 2 ml of catalyst stock solution and 1 ml of acetic acid-d₄ were added to a 4 ml thick-walled glass bulbs equipped with a PTFE valve. The vessel was then placed in a preheated oil bath at 170° C., and the reaction was monitored over time by GC-MS to determine the extent of H/D exchange. A control reaction lacking catalyst was also prepared and heated under identical conditions to correct for any background reaction. There was no observable H/D exchange between benzene and acetic acid lacking catalyst. TON was calculated as: (moles of D₁*1+moles of D₂*2+moles of D₃*3+ . . . )/moles of catalyst. Results are collected in Table 1.

### Example 13

### Conversion of Methane to Methanol Catalyzed by 1-TFA

A resealable metal reactor with a glass insert and stir bar was loaded with 90 mg of NaIO₄ (0.4207 mmol) and then 10.5 mg of 1-TFA (0.01487 mmol) in 1 ml of trifluoroacetic acid-d1 was added under argon. While stirring, the reactor was charged with 500 psi of methane. A control reaction was also prepared under identical conditions without 1-TFA catalyst. The reactors were then heated together at 180° C. for 3 h. The reactors were then cooled to room temperature and the head space was transferred to a 1 ml evacuated vial w/septa and analyzed by GC-MS which showed no detectable H/D exchange, possibly indicating that an Ir—CH₃ intermediate is efficiently trapped by periodate before exchange occurs.

The reactors were then opened and 10 μL of acetic acid was then added, there was no visible sign of decomposition. ¹H NMR analysis of the solution showed CH₃TFA formation in both reactions, indicating a slight background reaction between 104- and CH₄ in CF₃CO₂H. The yield of CH₃TFA in the presence of the (NNC)Ir is approx twice that in the absence of the catalyst. CH₃TFA formation was also supported by comparison to authentic sample by ¹³C NMR, GC-MS, and HPLC.

The conversion of methane to methanol in the 1-TFA catalyzed experiment was quantified as follows: amount of methanol produced 0.0936 mmol; number of turnovers (TON) 6.3; turnover frequency=6×10⁻⁴ (sec⁻¹).

### Example 14

### Octane Activation Catalyzed by 1-TFA

A solution of Ir(NNC)Et(TFA)(C₂H₄), 1-TFA (7.2 mg, 0.0102 mmol) in octane/trifluoroacetic acid-d1 (0.1 ml/0.9 ml) was heated at 160° C. After 0.5 hrs, 18.7% of n-octane was converted to deuterated isotopologues using ¹H NMR with trimethylcyclohexane as an internal reference. Extent of H/D incorporation was also followed by GC-MS, (Table 2) and loss of the parent ion peak at 114 amu, as well as the increase of the parent ion peak at 115 amu, support the formation of deuterated octane with catalyst 1-TFA present.

TABLE 2
GC/MS data for octane activation showing deuterium incorporation.

| m/z | | | | |
|---|---|---|---|---|
| 114 | 115 | 116 | 117 | 118 |

Octane blank

| | | |
|---|---|---|
| Relative | 92.23% | 7.77% |
| intensity | | |

Octane control at 160° C. 30 min

| | | |
|---|---|---|
| Relative | 92.27% | 7.73% |
| intensity | | |

Ir(NNC)Et(TFA)(C₂H₄), 1-TFA, 11.33 mM, at 160° C. 30 min

| | | | | | |
|---|---|---|---|---|---|
| Relative | 61.62% | 26.86% | 8.62% | 2.36% | 0.54% |
| intensity | | | | | |

### Example 15

### Cyclohexane Activation Catalyzed by 1-TFA

A solution of Ir(NNC)Et(TFA)(C₂H₄), 1-TFA (8.3 mmol) in cyclohexane/trifluoroacetic acid-d1 was heated at 160° C. After 6.5 hrs, cyclohexane was converted to deuterated isotopologues with catalyst present and analyzed using ¹H NMR with trimethylcyclohexane as an internal reference. Extent of H/D incorporation was also followed by GC-MS, (Table 3) and loss of the parent ion peak at 84 amu, as well as the increase of m/z ion peaks corresponding at 115 amu, support the formation of deuterated cyclohexane with catalyst 1-TFA present.

TABLE 3
GC/MS data for cyclohexane activation showing deuterium incorporation.

| m/z | | | | | | | | | |
|---|---|---|---|---|---|---|---|---|---|
| 84 | 85 | 86 | 87 | 88 | 89 | 90 | 91 | 92 | 93 |

Cyclohexane blank

| | | | |
|---|---|---|---|
| Relative | 94.0 | 5.8 | 0.2 |
| intensity | | | |

Cyclohexane control

| | | | |
|---|---|---|---|
| Relative | 94.0 | 5.8 | 0.2 |
| intensity | | | |

Ir(NNC)Et(TFA)(C₂H₄) 8.3 mM, 160° C., 6.5 h

| | | | | | | | | | | |
|---|---|---|---|---|---|---|---|---|---|---|
| Relative | 2.5 | 10.9 | 21.4 | 25.0 | 20.3 | 12.2 | 5.5 | 1.9 | 0.3 | 1.2 |
| intensity | | | | | | | | | | |

We claim:

1. A catalyst composition having formula: 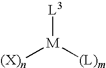
wherein:
M is osmium or iridium;
L³ represents an NNC tridentate ligand wherein two nitrogen donor atoms and one carbon donor atoms covalently bind M;
X represents a formal anionic ligand and n is 1 to 2;
L represents a formal neutral ligand and m is 0 to 1.

2. The composition of claim 1 wherein L³ has molecular formula: 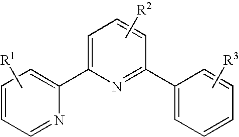
wherein R¹, R², R³ are each independently selected from the group consisting of hydrogen, optionally substituted C₁-C₅ linear or branched alkyl, amine (—NHR¹), amino, hydroxy, or optionally substituted C₁-C₅ alkoxy.

3. The composition of claim 1 wherein L is nitrile, alkene, or solvent and m is 1.

4. The composition of claim 1 wherein each X is independently selected from the group consisting of alkyl, halide, optionally substituted carboxylate, sulfate, and optionally substituted sulfonate; and n is 2.

5. A catalyst composition according to claim 1 comprising structure: 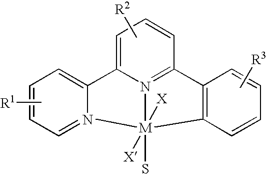
wherein:
M is osmium or iridium;
R¹, R², R³ are each independently H, optionally substituted C₁-C₅ branched or linear alkyl, amine (—NHR¹), amino, hydroxy, or optionally substituted C₁-C₅ alkoxy;
X and X′ are each independently selected from the group consisting of optionally substituted C₁-C₂ alkyl, aryl, halide and carboxylate;
S is alkene, solvent or nitrile.

6. A composition of claim 5 wherein R¹ and R² are both branched C₁-C₅ alkyl, and R³ is H.

7. A composition of claim 1 wherein L is nitrile or alkene.

8. The composition of claim 7 wherein each X is independently selected from the group consisting of alkyl, halide, and optionally substituted carboxylate.

9. A catalyst composition according to claim 1 comprising 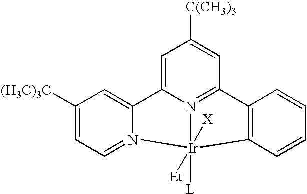
wherein:
X is selected from the group trifluoroacetate, chloride, acetate;
L is nitrile or alkene.

10. A CH bond activation process comprising contacting one or more alkane or arene hydrocarbon with a catalyst composition of claim 1 under functionalizing conditions, wherein said activation process may be optionally detected using isotope labeling.

11. A process according to claim 10 wherein the hydrocarbon is methane or benzene.

12. A process according to claim 10 wherein the catalyst composition comprises a carboxylic acid solvent.

13. A process according to claim 12 wherein the carboxylic acid solvent is selected from the group consisting of acetic acid, trifluoroacetic acid.

14. A process according to claim 12 wherein the catalyst composition further comprises an oxidant.

15. A process for the selective oxidation of alkane and arene hydrocarbons comprising the steps:
a) contacting the hydrocarbon and oxidant with a catalyst of structure: 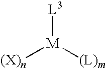
wherein:
M is a transition metal selected from group 8 and group 9 of the periodic table;
L³ represents an NNC tridentate ligand wherein two nitrogen donor atoms and one carbon donor atoms covalently bind M;
X represents a formal anionic ligand;
n is 1 to 2;
L represents a formal neutral ligand;
m is 0 to 1.

16. The process of claim 15 wherein M is osmium or iridium.

17. The process of claim 15 wherein L³ is 6-phenyl-4,4′-bipyridine or a substituted derivative thereof.

18. The process of claim 15 wherein L³ is nitrile or alkene and m is 1.

19. The process of claim 15 wherein n is 2 and each X is independently selected from the group consisting of alkyl, halide, and carboxylate.

20. A process for the selective oxidation of a hydrocarbon, comprising:
passing a feed comprising hydrocarbon and an oxidant to a first catalyst zone comprising a metal catalyst of claim 1, at functionalization conditions, to form an effluent comprising oxygenated hydrocarbon product and reduced oxidant;
separating the oxygenated hydrocarbon product from the reduced oxidant;
passing the reduced oxidant and a reoxidizer to a reoxidation zone, at reoxidizing conditions, to reform the oxidant;
wherein the metal catalyst comprises iridium, and where the metal is coordinated to a NNC tridentate ligand, and wherein the functionalization conditions comprise a temperature of between 100 and 350 degrees C. and a solvent having an acidity level selected from the group consisting of neutral and acidic.

21. The process of claim 20 wherein the feed comprises an alkane and the oxygenated hydrocarbon product comprises an alcohol.

22. The process of claim 20 wherein the feed comprises methane and the product oxygenated hydrocarbon comprises methanol.

23. The process of claim 20 wherein the first catalyst zone further comprises a solvent selected from the group consisting of an acid or a neutral solvent.

24. The process of claim 20 wherein the functionalization conditions comprise a temperature of between 150 and 250 degrees C.

25. The process of claim 20 wherein the reoxidizer is oxygen.

26. The process of claim 20 wherein the reoxidizer is air.

27. The process of claim 20 wherein the activated metal catalyst is supported on a solid support.

28. The process of claim 20 wherein the oxidant is an O-atom donor.

29. The process of claim 20 wherein the O-atom donor is selected from the group consisting of cupric oxide (CuO), selenate, (SeO₄²⁻), vanadate (VO₄³⁻), and sulfoxide.

30. A hydrocarbon conversion process, comprising:
passing a feed comprising hydrocarbons to a CH activation zone, comprising a CH activation catalyst and a solvent, at CH activation conditions, to form an activated hydrocarbon;
contacting the activated hydrocarbon with a functionalizing agent to produce a functionalized hydrocarbon; and
wherein the CH activation zone comprises a solvent having an acidity level selected from the group consisting of neutral, acidic and highly acidic, the catalyst comprises one or more transition metal selected from the group consisting of Re, Os, Ir, and one or more ligand having an NNC configuration.

31. The process of claim 30 wherein the feed comprises an alkane and the functionalized hydrocarbon is an alcohol.

32. The process of claim 30 wherein the alcohol is methane.

33. The process of claim 30 wherein the feed comprises an arene and the functionalized hydrocarbon is a phenol.

34. The process of claim 33 wherein the feed is benzene.
